(19) United States
(12) Patent Application Publication
(10) Pub. No.: US 2005/0049540 A1
(43) Pub. Date: Mar. 3, 2005

(54) Monoshunt

(75) Inventor: Louis P. Perrault, Outremont (CA)

Correspondence Address: Mr. James A. Sheridan;PANDISCIO & PANDISCIO, P.C., 470 Totten Pond Road, Waltham, MA, 02451-1914, US

(73) Assignee: INSTITUT DE CARIOLOGIE DE MONTREAL/MONTREAL HEART INSTITUTE

(21) Appl. No.: 10/841,400

(22) Filed: May 7, 2004

Related U.S. Application Data
(60) Provisional application No. 60/468,269, filed on May 7, 2003.

(51) Int. Cl. A61B 17/08

(52) U.S. Cl. 604/8

(57) ABSTRACT

Insertion of intracoronary shunts during off-pump coronary artery bypass (OPCAB) surgery can induce a severe endothelial dysfunction. The present invention relates to a novel intracoronary shunt design and the use of this device during surgery as a means to prevent this undesirable effect. Termed the “Monoshunt”, this intracoronary shunt advantageously avoids distal endothelial denudation and allows distal coronary perfusion.

### FIELD OF THE INVENTION

The present invention relates to a novel intracoronary shunt design. Termed the “Monoshunt”, this intracoronary shunt avoids distal endothelial dysfunction during off-pump coronary artery bypass (OPCAB) surgery.

### BACKGROUND OF THE INVENTION

Off-pump coronary artery bypass (OPCAB) surgery has regained popularity in recent years [1] with a reduction of morbidity for selected patients and significant decreases in cost compared with conventional on-pump surgery in some series [2]. However, specific technical difficulties are associated with this approach, such as heart stabilization, coronary bleeding at the anastomotic site, or the maintenance of distal perfusion during coronary occlusion.

The insertion of intracoronary shunts has been used in coronary artery bypass grafting surgery since 1975 [3]. This hemostatic system has the double advantage of drying the anastomotic site (hemostatic effect) while allowing an effective distal coronary perfusion (myocardial protection), which may sometimes be necessary in off-pump coronary artery bypass (OPCAB) surgery. Studies of the effects of intracoronary shunts on the endothelium of porcine coronary arteries have demonstrated deleterious consequences on endothelium-dependent reactivity [4], due to the rubbing of the shunt on the endothelial layer. Distal endothelial lesions and dysfunction are particularly worrisome because they may involve the distal run-off of the bypass.

The necessity of a bloodless field to obtain optimal visibility during performance of the anastomosis is an issue of concern in OPCAB. The most widely used variant of OPCAB involves use of sutures or silastic tapes to snare the coronary artery extravascularly, upstream and downstream from the anastomotic site on the target artery. However, examination with scanning electron microscopy has shown that snares cause focal endothelial denudation, microthrombosis, and atherosclerotic plaque rupture [6] which may have severe clinical consequences, especially in diabetic patients [7].

Intracoronary shunts used as hemostatic devices in OPCAB also have the advantage of allowing myocardial protection by maintaining distal coronary perfusion. Experimental [8] and clinical studies [9] have demonstrated that shunting can prevent acute left ventricular dysfunction during beating heart coronary revascularization and is a useful tool in patients with left ventricular dysfunction or unstable angina, as well as for teaching OPCAB to residents [10]. However, shunts cause a severe endothelial dysfunction [4] due to rubbing of the endothelial layer [11] during the positioning and the removal of the devices. This can acutely compromise the patency of the bypasses and contribute to late graft failure and recurrent angina by favoring the development of intimal hyperplasia.

There is therefore a need for a shunt that avoids severe endothelial dysfunction.

### SUMMARY OF THE INVENTION

The present invention seeks to meet this need. Specifically, the present invention relates to a novel intracoronary shunt design called Monoshunt that avoids distal endothelial damage of a target coronary blood vessel.

In one embodiment of the present invention, the Monoshunt comprises:
a body having a blood flow lumen therethrough, the blood flow lumen having first and second openings for passing blood from one side of an opening in the blood vessel to a second side of the opening in the blood vessel; and
an occluding member attached to the body and being sized and configured to occlude the blood vessel on a single side of the opening in the blood vessel.

In an alternative embodiment of the present invention, the Monoshunt comprises a T-shaped shunt adapted to be inserted and removed through an incision in the blood vessel and including:
an elongated primary perfusion tube having a first open end, a second open end and a central passage extending between and interconnecting the open ends;
a secondary perfusion tube intersecting the primary perfusion tube and having a first open end and a second open end intersecting with the primary tube and having a central passage extending between the open ends; and
an enlarged occluding member (also called occluder) adjacent one open end of the primary tube to seal the interior of the blood vessel, the primary perfusion tube passage allowing blood flow through the vessel during cardiac or vascular procedures.

The Monoshunt differs from commercially available shunts by having a single occluder or bulb, as opposed to the standard two. Surprisingly, the use of the Monoshunt with an undersized and flexible distal part avoids rubbing of the device on the endothelial layer and, as a result, the occurrence of endothelial dysfunction in the distal run-off. Advantageously, hemostasis has been found to be satisfactory with the Monoshunt, allowing for the completion of an anastomosis more expediently than has heretofore been possible.

The present invention further includes the use of the Monoshunt during surgery.

Other objects, advantages and features of the present invention will become more apparent upon reading of the following non restrictive description of preferred embodiments thereof, given by way of example only with reference to the accompanying drawings.

### BRIEF DESCRIPTION OF THE DRAWINGS

FIG. 1: An embodiment of the Monoshunt: 2.5 mm diameter of the proximal occluder (Clearview®, Medtronic, Grand Rapids, Mich., USA). Shunt length 20 mm. U; upstream, D; downstream.

FIG. 2: A) and B) illustrate alternative embodiments of the Monoshunt shown in FIG. 1.

FIG. 3: A schematic view of a patient for coronary bypass surgery with positions for incisions in the chest wall indicated for thorascopic bypass grafting.

FIG. 4: A schematic showing of the thoracoscopic instruments extending through small incisions or ports in the chest wall.

FIG. 5: A) Schematic view of the Monoshunt before insertion into an incision; B) Schematic view similar to A), but with the Monoshunt inserted into the incision in the artery; C) Schematic view similar to B), but showing the graft partially sutured to the artery.

FIG. 6: An elevation view of a patient's heart with a Monoshunt which embodies features of the invention in place on the patient's heart during perfusion of a coronary artery.

FIG. 7: Illustration of the Monoshunt shown in FIG. 5, after the Monoshunt is positioned proximally within the blood vessel.

FIG. 8: Illustration of the Monoshunt shown in FIG. 6, during suturing of a bypass graft to the blood vessel.

FIG. 9: Illustration of the Monoshunt shown in FIG. 8, nearing the completion of suturing of the bypass graft to the blood vessel.

FIG. 10: Cumulative concentration-contraction response to prostaglandin F_{2≡} (PGF_{2≡}) in porcine right coronary arteries rings. Submitted to the Monoshunt and in control rings. A P-value less than 0.05 was considered statistically significant.

FIG. 11: A) Cumulative concentration-relaxation response curves to serotonin (5-HT) in porcine right coronary arteries rings submitted to the Monoshunt and in controls rings; B) Cumulative concentration-relaxation response curves to bradykinin (BK) in porcine right coronary arteries rings submitted to the Monoshunt and in control rings; C) Cumulative concentration-relaxation response curves to sodium nitroprussiate (SNP in porcine right coronary rings submitted to the Monoshunt and in control rings). A P-value less than 0.05 was considered statistically significant.

FIG. 12: Coronary artery Silver Nitrate staining A) showing a preserved endothelial layer, B) Showing preservation of the endothelial layer with the distal part of the Monoshunt (50% to 100% of controls); C) Showing disappearance of the endothelial layer due to the rubbing with the proximal part of the Monoshunt (0% of controls).

### DESCRIPTION OF THE PREFERRED EMBODIMENTS

The Monoshunt generally includes a primary elongate tubular member that is sized and dimensioned to be inserted into a target vessel, such as the right coronary artery.

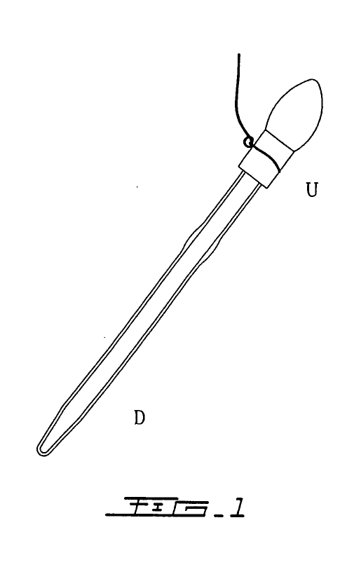

In one embodiment of the Monoshunt, one end of a standard commercially available intracoronary shunt 2.5 mm diameter (Clearview®, Medtronic, Grand Rapids, Mich., USA) was cut off to obtain an isolated occluder. The distal part of an intravenous catheter (Cathlon®, Johnson and Johnson, Arlington, Tex., USA) was cut to obtain a tube 2 cm in length and 1.8 mm in external diameter, which was imbricated into the occluder to obtain the Monoshunt (FIG. 1).

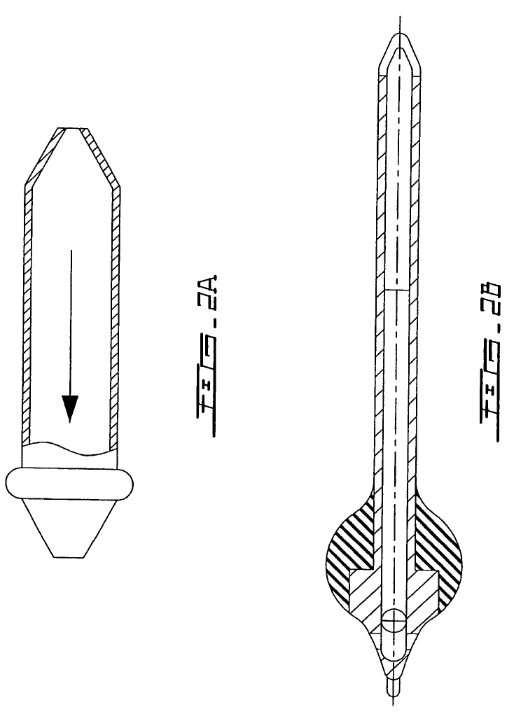

As will be understood by one of skill in the art, other commercially available intracoronary shunts of various shapes and diameters may be similarly modified to suit specific surgical requirements. For example, FIG. 13 illustrates two alternative embodiments of the Monoshunt shown in FIG. 2. To avoid endothelial damage, the selected intracoronary shunt is an undersized shunt with minimal endothelial rubbing associated with the obligatory shunt's movement for positioning and removal, which is the most deleterious maneuver for the endothelium. However, if endothelial damage remains inevitable upstream, use of a Monoshunt as described herein should avoid distal endothelial denudation, protect the run-off of the bypass and allows distal coronary perfusion.

Generally, the Monoshunt comprises the following features:
A body having a blood flow lumen therethrough, the blood flow lumen having first and second openings for passing blood from one side of an opening in the blood vessel to a second side of the opening in the blood vessel; and
An occluding member attached to the body and being sized and configured to occlude the blood vessel on a single side of the opening in the blood vessel.

Additional features may be selected from the following non-exhaustive list:
An occluding member that is slightly greater in diameter than the exterior diameter of said body;
A transparent body;
An occluding member at the end of the Monoshunt's body that is integral with it;
Selection of the diameter of the body of the Monoshunt's body so as to be slightly inferior to the diameter of the target blood vessel; and
Construction from a biocompatible material chosen from polyethylene, polyurethane, nylon, silicone, or other suitable single or composite material.

In an alternative embodiment, the Monoshunt comprises a T-shaped shunt adapted to be inserted and removed through an incision In the blood vessel which includes:
An elongated primary perfusion tube having a first open end, a second open end and a central passage extending between and interconnecting the open ends;
A secondary perfusion tube intersecting the primary perfusion tube and having a first open end and a second open end intersecting with the primary tube and having a central passage extending between the open ends; and
An enlarged occluding member adjacent one open end of the primary tube to seal the interior of the blood vessel, such that the primary perfusion tube passage allows blood flow through the vessel during cardiac or vascular procedures.

Additional features for this alternative embodiment may be selected from the following non-exhaustive list:
The secondary perfusion tube passage intersects the primary perfusion tube passage for fluid communication therewith;
The secondary perfusion tube allows connection to an appropriate blood supply;
The occluding member is slightly greater in diameter than the exterior diameter of said primary perfusion tube;
A Y-connector may be inserted in the secondary perfusion tube to provide a direct line and a second line from the Y-connector for a needle-less valve;
The primary and secondary perfusion tubes are unitary thin wall tubes, with the primary tube being of a slightly smaller exterior diameter than the interior diameter of the blood vessel;
The primary and secondary perfusion tubes are transparent;
The occluding member at the end of the primary perfusion tube is integral with it;
The shunt provides a dual perfusion, completely open system;
The diameter of the body of the Monoshunt is selected so as to be slightly inferior to the diameter of the target blood vessel; and
The intracoronary shunt is constructed from a biocompatible material chosen from polyethylene, polyurethane, nylon, silicone, or other suitable single or composite material.

Methods for using the Monoshunt are also described which generally include making an incision in the target vessel and inserting the proximal and distal ends of the primary tubular member of the Monoshunt into the target vessel via the incision. The Monoshunt is suitable for a number of coronary procedures, including anastomosis and bypass surgery. For example, in the case of coronary bypass surgery, the Monoshunt may be used for retaining blood flow through a blood vessel. This includes the steps of locating the blockage in a blood vessel necessitating bypass surgery, making an incision adjacent the blockage, inserting the Monoshunt into the blood vessel to retain the vessel open and ensuring that the occluder is positioned to prevent leakage of blood around the shunt, suturing a vein graft onto the outline of the incision, and gradually removing the intracoronary shunt as the suturing of the graft to the blood vessel is completed.

Additionally, use of the Monoshunt during surgery allows the following:
Insertion of drugs into the shunt via the secondary tube (where such secondary tube exists) during the procedure. The secondary tube may extend to the exterior of the patient's body from the primary perfusion tube for insertion of drugs or blood from a secondary source;
Additional blood may be pumped from another area of the patient's body or from an external pump through the secondary perfusion tube;
Coronary bypass procedure may be accomplished with thoracoscopic instruments involving several small ports resulting in a minimally invasive coronary bypass surgery; and
The intracoronary shunt may be capable of insertion into the chest cavity of the patient through a small incision for endoscopic surgery.

### EXAMPLE 1

### Use of the Monoshunt

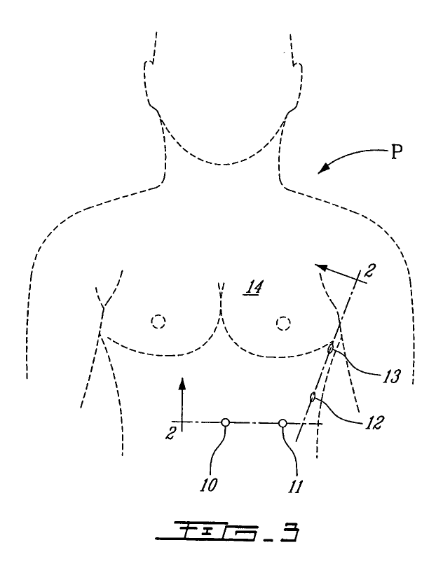

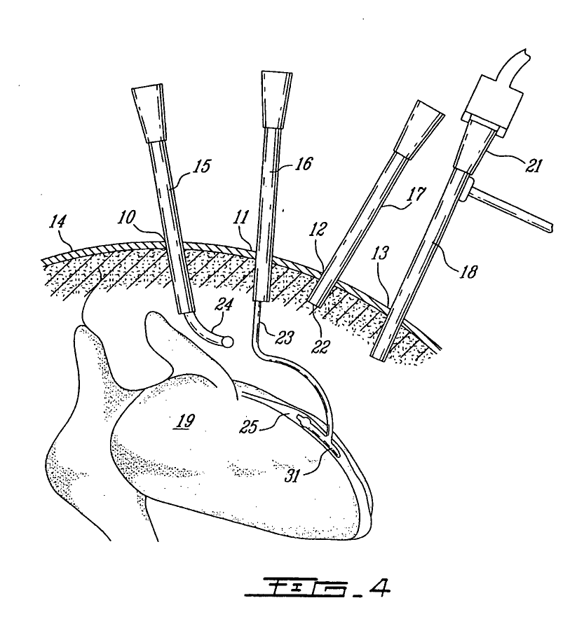

Referring more particularly to the disclosure in the drawings wherein are shown illustrative embodiments of the present invention, FIG. 3 discloses a candidate or patient P for coronary bypass surgery with the location of four incisions 10, 11, 12 and 13 in the patient's chest wall 14 shown for use of endoscopic instruments 15, 16, 17 and 18 (see FIG. 4). Unlike previous coronary bypass surgery where the heart is stopped and the patient is kept alive by the circulation of his blood to the brain and vital organs provided by a heart/lung machine, the Monoshunt 31 of the present invention allows the heart 19 to remain beating with blood flow through the Monoshunt.

As shown in FIG. 4, thoracoscopic instruments 15, 16, 17 and 18 are inserted through the incisions or ports 10, 11, 12 and 13 in the chest wall 14 for access to the patient's heart 19. These instruments may include a thoracoscopic camera and fiber optic light 21, endoscope 22, instrument 23 to guide and manipulate the Monoshunt 31, and instruments 24 for operating on the target vessel.

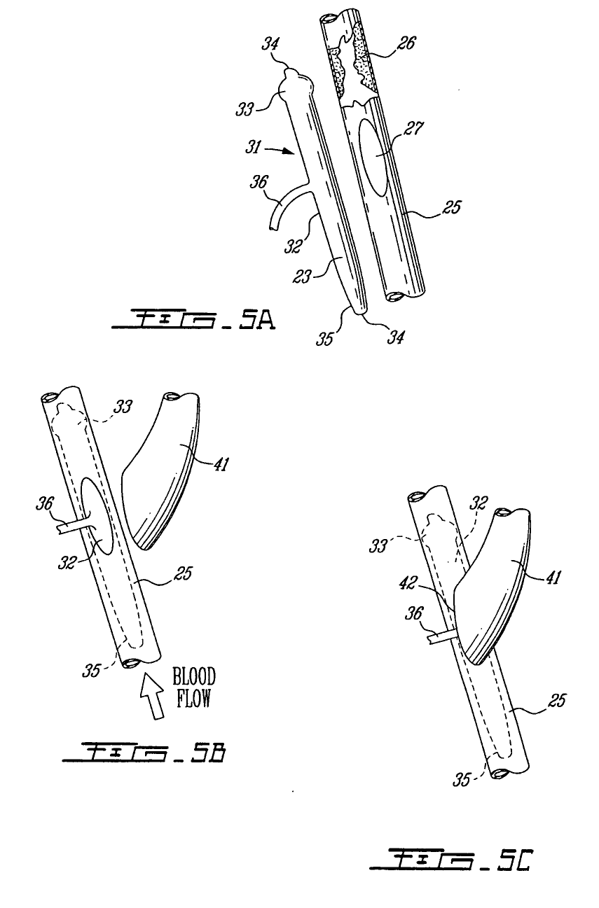

Referring now to FIG. 5, once a blockage 26 of the target vessel 25 is located, an incision 27 is made adjacent to the blockage 26 which is of sufficient length to allow insertion of the Monoshunt 31 into the vessel. As shown in FIG. 5A), the Monoshunt 31 is formed as a short length of thin wall member or primary perfusion tube 32 having an enlarged occluding member (also called occluder) or bulb 33 at one end 34 of the tube, with the opposite end 34 of the tube having beveled surface or tip 35. Between the ends of the primary tube 32 is a secondary perfusion tube 36 intersecting the primary tube at an angle of approximately ninety degrees (right angle). In practice, the primary tube is of a length of approximately 2.0 centimeters (cm.) in length, while the secondary tube has a length of approximately 10.0 cm. (or 25 cm.).

To properly size the appropriate Monoshunt for the vessel, various sizes of Garrett probes can be inserted into the blood vessel containing the blockage 26. The appropriate Monoshunt and occluder are selected from the diameter of probe found to be appropriate for the vessel.

The Monoshunt 31 allows blood flow through the target vessel as a graft 41 is sewn onto the incision 27 in the artery and keeps the artery open (FIGS. 58 and 5C). Thus, by allowing blood flow and preventing backbleedlng due to the occluder or bulb 33, the Monoshunt increases safety of the coronary bypass operation by allowing sufficient time for suturing the graft 41 onto the incision 27 to reduce the stress on the surgeon performing the operation, provide reproducibility of results from patient to patient and reduce the possibility of ischemic reactions during and after the operation. As the external diameter of the primary tube is smaller than the internal diameter of the blood vessel, suitable spacing is provided between the primary tube and vessel wall to allow the sliding of the sutures into the vessel wall to attach the graft to the incision. As the suturing of the graft 41 at 42 onto blood vessel at the incision nears completion, the Monoshunt 31 is gradually withdrawn through the incision by traction on the side limb or secondary tube 36, the final sutures are completed and the suture ends are tied.

### EXAMPLE 2

### Use of the Monoshunt in an Anastomosis Procedure

The Monoshunt 31 can be used in a method for performing a medical procedure, such as perfusion of a blood vessel during an anastomosis.

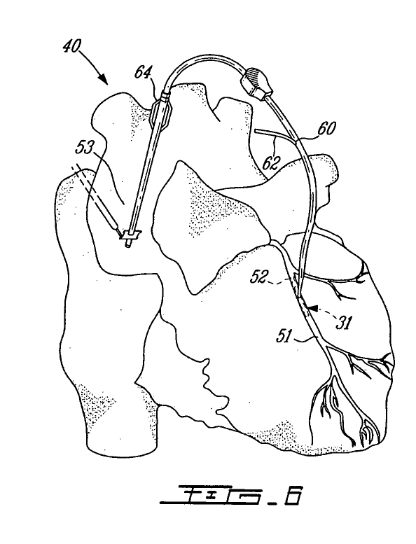

FIG. 6 illustrates a patient's heart 40 with a Monoshunt 31 in place during perfusion of a coronary artery. The distal section of the Monoshunt's shaft 32 is within a coronary artery 51 distal to a lesion 52 therein, and the proximal end of the needle adapter 64 is within the ascending aorta 53.

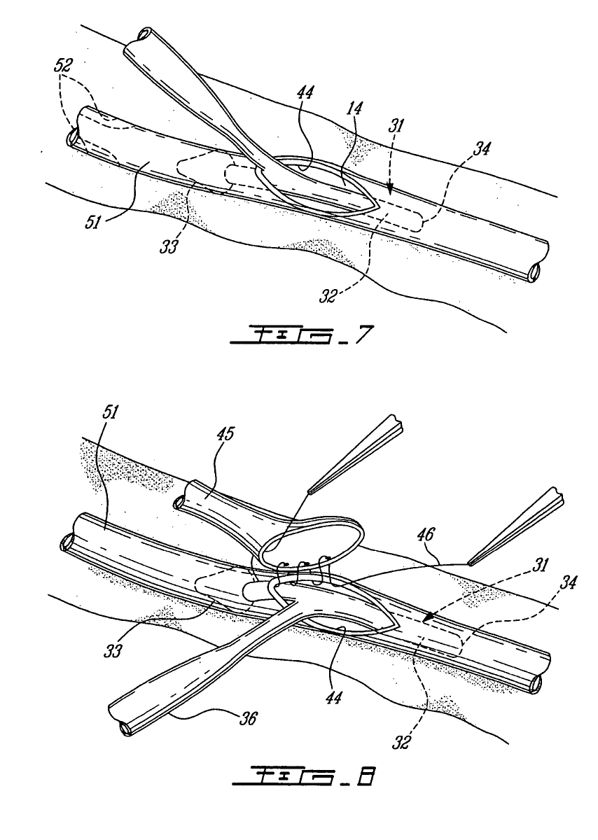

FIGS. 7-9 illustrate the performance of an anastomosis at the site of the distal section of the shaft 32 of the Monoshunt 31. As illustrated in FIG. 7, the distal section of the shaft 32 has been inserted through a surgical incision 44 (arteriotomy) or other opening in the coronary artery 51. The Monoshunt 31 is inserted, displaced proximally and anchored within the artery 51. The anchored distal section of the shaft 32 prevents or inhibits the proximally thrusting force of the perfusion fluid flowing distally out the port 34 from causing further displacement of the distal shaft section proximally within the artery 51 and out of the incision 44 during perfusion. With the distal section of the shaft 32 in place within the artery 51, perfusion is started by connecting the proximal end of the Monoshunt to an arterial line, as by inserting the needle adapter 64 into the aorta 53 (see FIG. 6) and allowing blood flow from the aorta, into the adapter 64 and lumen 23 in fluid communication therewith, and out the port 34 in the distal end of the distal section of the shaft 32 within the coronary artery 51.

The distal occluder 33 prevents or inhibits the flow of blood or perfusion fluid around the outer surface of the distal section of the shaft 32 in the artery 51, and has an outer diameter at a maximum dimension which is configured to avoid contact with the inner surface of the coronary artery. As a result, visualization of the anastomosis site is facilitated during attachment of a graft vessel to the coronary artery 51 at the incision 44.

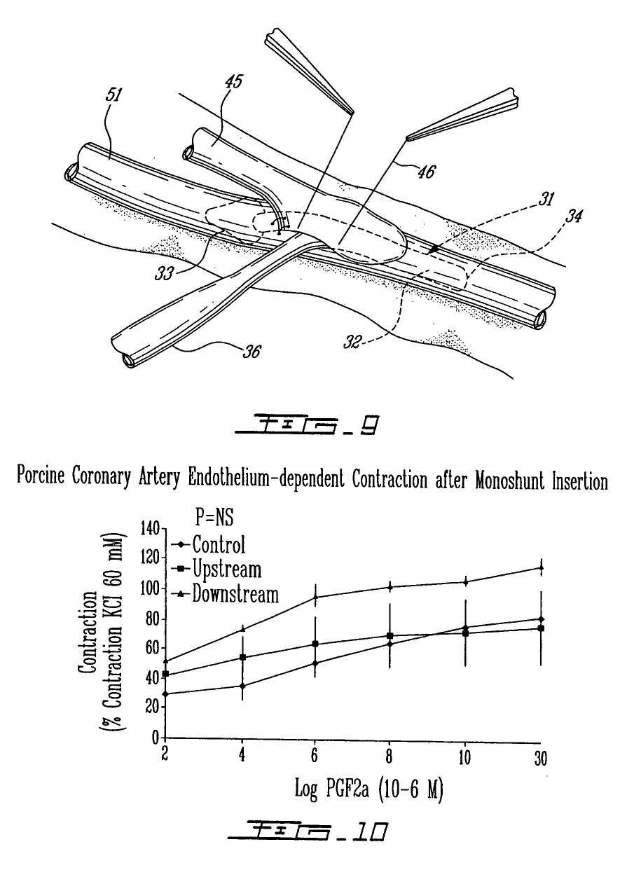

FIG. 8 illustrates a graft vessel 45 being sutured to the artery 51 with suture 46 around the incision 44, with the Monoshunt 31 perfusing the myocardium distal to the incision 44 during the suturing. The expression “graft vessel” should be understood to include a variety of conventional implants including synthetic and natural prostheses, grafts and the like. Graft vessel 45 may comprises a variety of suitable materials, as are conventionally used in anastomosis procedures, including natural and synthetic materials, such as heterologous tissue, homologous tissue, polymeric materials, Dacron, and fluoropolymers, and polyurethanes, and the like.

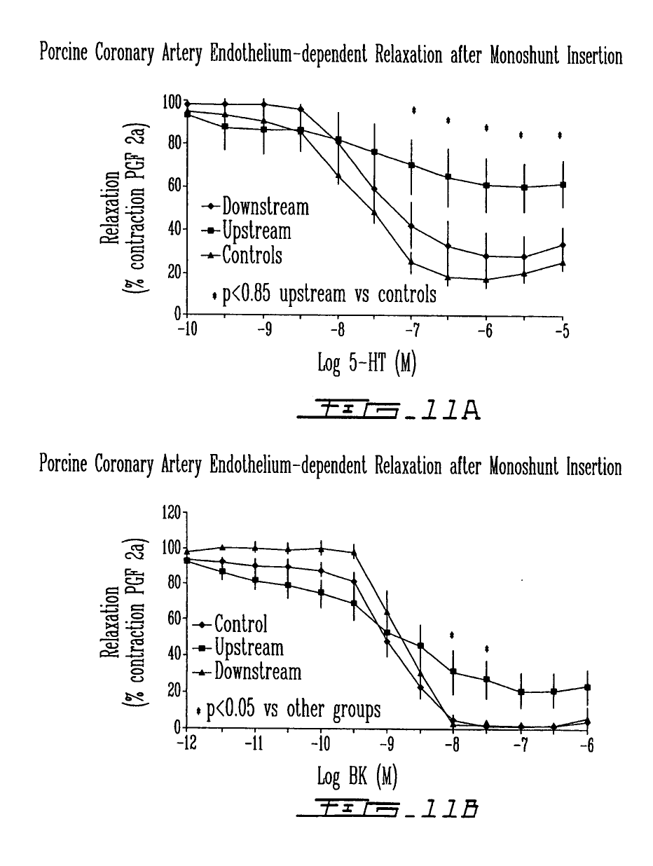

FIG. 9 illustrates the graft vessel 45 sutured to the artery 51 before the Monoshunt 31 is removed and the final sutures are tied. The perfusion flow is stopped, and the Monoshunt 31 is pulled proximally to remove the distal section of the shaft 32 from the coronary artery 51 and that exit is sutured to complete the suturing of the graft vessel 45 to the artery 51. The bypass surgery is completed by anastomosis of the other end of the graft vessel 45 to an artery section proximal to the lesion 52.

### EXAMPLE 3

### Experimental Surgery

Six white Landrace swine of either gender, aged 8±1 weeks and weighing 24.9±4.0 kg were selected for experimental surgery. Animals were maintained and tested in accordance with the recommendations of the Guidelines on the Care and Use of Laboratory Animals issued by the Canadian Council on Animals. The animals were sedated with an intramuscular injection of 25 mg/kg of ketamine hydrochloride (Ayerst Veterinary Laboratories, Guelph, ON, Canada) and 10 mg/kg of xylazine (Boehringer Ingelheim, Burlington, ON, Canada), intubated and mechanically ventilated with an oxygen/air mixture (3:2). Anesthesia was maintained with 1 to 2.5% halothane inhalation (Halocarbon Laboratories, River Edge, N.J., USA). The electrocardiogram was recorded from three subcutaneous limb electrodes. The heart was then exposed via a median stemotomy approach and 300 U/kg heparin (Leo Pharma, Inc., Ajax, ON, Canada) were given intravenously. The Monoshunt was then inserted via a 5-mm longitudinal arteriotomy on the proximal part of the right coronary artery (RCA). It was inserted first downstream to the arteriotomy and then proximally to position the shunt's occluder. The Monoshunt was left in place for 15 mm and bleeding at the anastomotic site was measured semiquantitatively (+++: impossible anastomosis, ++: possibility of anastomosis despite bleeding, +: very little bleeding and 0: no bleeding) [5]. The flow through the Monoshunt was measured by quantification of the quantity of blood per mm. The Monoshunt was then removed and the heart was excised and placed in a modified Krebs-bicarbonate solution (composition in mmol/l: NaCl 118.3, KCl 4.7, MgSO₄ 1.2, KH₂PO₄ 1.2, glucose 11.1, CaCl₂ 2.5, NaHCO₃ 25, and EDTA 0.026).

Functional Coronary Testing

Coronary arteries were dissected free of the fatty epicardial tissue in a Petri dish filled with oxygenated modified Krebs-bicarbonate and were divided into rings 5 mm in length. Two instrumented rings were obtained from the RCA, upstream (proximal) and downstream (distal) from each arteriotomy at the site of the Monoshunt positioning. Control rings were obtained from non-instrumented coronary arteries. All rings were placed in organ chambers (Emka Technologies Inc., Paris, France) filled with 20 ml modified Krebs-bicarbonate solution heated at 37° C. and oxygenated with a carbogen mixture (95% 02 and 5% CO₂). The rings were suspended between two metal stirrups with the upper one connected to an isometric force transducer, and then allowed to stabilize for 30 mm. Data were collected with a biological signal data acquisition software (IOX 1.203; Emka Technologies Inc., Paris, France).

Each arterial ring was stretched to the optimal point of its active length-tension curve (approximately 3.5 g). The maximal contraction of rings was then obtained with addition of potassium chloride (KCl 60 mmol/l). After obtention of a plateau, all baths were washed twice with modified Krebs-bicarbonate solution and indomethacin (10⁻⁵ mol/l to exclude production of endogenous prostanoids), propranolol (10⁻⁷ mol/l to prevent the activation of 3-adrenergic receptors) and ketanserin (10⁻⁸ mol/l to block serotonin 5-HT₂ receptors) were added in each bath.

After 45 mm of stabilization, prostaglandin F_{2≡} (range 2×10⁻⁶ to 3×10⁻⁵ mol/l) was added to obtain a contraction averaging about 50% of the maximal contraction to KCl. Endothelium-dependent relaxations to serotonin (5-hydroxytryptamine creatine sulfate: 5-HT; an agonist which binds to 5-HT_{ID} receptors coupled to Gᵢ-proteins) at incremental concentrations (10⁻¹⁰ to 10⁻⁵ mol/l) and bradykinin (BK; an agonist which binds to B₂ receptors coupled to Gq-proteins) at various concentrations (10⁻² to 10⁻⁸ mol/l) were quantified.

Endothelium-independent relaxations were studied by constructing concentration-response curves to sodium nitroprusside (SNP, 10⁻¹⁰ to 10⁻⁵ mol/l, an exogenous NO donor).

Endothelium-dependent contractions were studied by constructing concentration-response curves to prostaglandin F_{2≡} (PGF_{2≡}, 2×10⁻⁸ to 3×10⁻⁵ mol/l).

Morphologic Coronary Examination

Segments of fresh instrumented and control coronary arteries were used for silver nitrate staining to visualize the remaining intact endothelium. Rings from each group (3 mm, 2 mm, 1.25 mm, controls) were opened longitudinally to obtain 4×8 mm strips and pinned to the bottom of a Petri dish filled with saline solution. The strips were first fixed for 10 mm with a phosphate buffer (0.1 mol/l) added with paraformaldehyde and glutaraldehyde. After a 1-mm wash with sucrose solution, 0.25% silver nitrate (Sigma Chemical Co., ON, Canada) was added, followed 1 mm later by a second washing during 1 mm. This was followed by a second fixation period during 2 mm and incubation was done in a sodium cacodylate solution under a UV spotlight exposure for 3 h. The stained specimen were mounted whole on glass slides and labeled. The percent surface area covered by intact endothelium was then estimated under microscope magnification (×250).

Statistical Analysis

All values are expressed as the mean±standard error of the mean (SEM). Contractions to prostaglandin F_{2≡} are expressed as a percentage of the maximal contraction to KCl (60 mmol/l).

Relaxations are expressed as the percentage of the maximal contraction to prostaglandin F_{2≡} for each ring. Two-way repeated analysis of variance (ANOVA) were performed to compare each point of the concentration-response curves between control rings and instrumented rings upstream and downstream from the anastomotic site. Statistical analysis was realized with the computer software S.A.S. (Insert Inc., Cary, N.C., USA). A P-value of less than 0.05 was considered statistically significant.

Results

Experimental Surgery

All Monoshunts were positioned into the right coronary artery after a single attempt and remained patent throughout the duration of the experiment. Insertion of the Monoshunts into the RCA and the Left Anterior Descending (LAD) was well tolerated hemodynamically during the whole experiment (data not shown). Hemostasis (0 or +) was always obtained at the arteriotomy site with the Monoshunt. The flow was 30 ml/mm under 55±10 mmHg of mean blood pressure (40 ml/min for the standard shunt).

Coronary Reactivity Study

Contractions

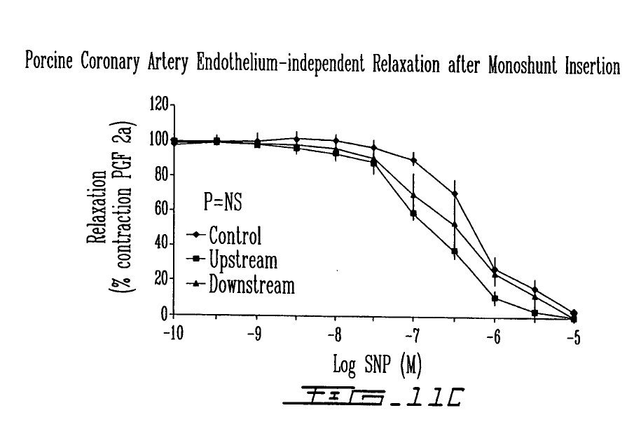

The amplitude of the contraction to KCl (60 mmol/l) and to prostaglandin F_{2≡} (2×1⁻⁶ to 3×10⁻⁵ mol/l) (FIG. 10) was quantified for all groups (upstream, downstream, controls) and there was no significant differences in contractions between the different groups.

Relaxations

Endothelium-Dependent Relaxations

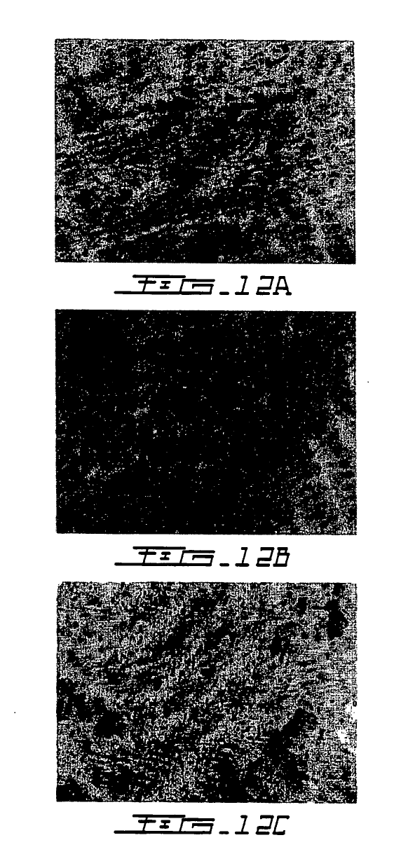

There was a statistically significant decrease (P<0.05) in endothelium-dependent relaxation to 5-HT in rings located upstream (occluder side) from the arteriotomies, compared with the control group. There was no statistically significant decrease of relaxations to 5-HT in rings located downstream (no occluder side) from the arteriotomies, compared with the control group (FIG. 11A).

There was a statistically significant decrease (P<0.05) in endothelium-dependent relaxation to BK in rings located upstream (occluder side) from the arteriotomies, compared with the control group. There was no statistically significant decrease of relaxations to BK in rings located downstream (no occluder side) from the arteriotomies, compared with the control group (FIG. 11B).

Endothelium-Independent Relaxations

No differences in endothelium-independent relaxations to the NO donor SNP were observed in coronary rings between groups (FIG. 11C).

Coronary Morphologic Study

All instrumented strips were compared with control strips (FIG. 12A). Histological study of the endothelial cell coverage demonstrated preservation of the endothelial layer with the distal part of the Monoshunts (90-100% of controls) (FIG. 12B), and a total disappearance of the endothelium (0% of controls) on strips instrumented with the proximal part of the Monoshunts (FIG. 12C).

The major conclusions to be drawn from the above are: there is marked decrease of endothelium-dependent relaxation due to the Monoshunt upstream from the arteriotomy, and no significant endothelial dysfunction downstream associated with no or minor bleeding allowing the performance of anastomosis. The upstream endothelial dysfunction involves Gi protein and Gq mediated endothelium-dependent relaxations suggestive of a severe endothelial dysfunction. Endothelium-independent relaxations were unaffected both upstream and downstream by the use of such shunts, demonstrating the integrity of the underlying smooth muscle cells.

The main limitation of this study is the use of healthy coronaries arteries, with a large and unimpaired run-off, and experiments should be repeated on atherosclerotic arteries [12] but the lack of rubbing by the distal part would also protect the endothelium in these arteries. Furthermore, as the endothelium of atheromatous arteries endothelium is already dysfunctional [13] the differences of effect of the Monoshunt between both sides of the anastomotic site would not appear as clearly in these experiments.

Finally, in chronically occluded vessels with a large collateral bloodflow and generous retrograde perfusion, back bleeding at the anastomotic site may be controlled imperfectly by the Monoshunt's distal area. However, these occluded vessels will seldom need shunting for myocardial protection.

As known to those of skill in the art, caution must be used in the application of the Monoshunt because all shunts can generate serious macroscopic complications such as extensive intimal denudation and atheromatous plaque rupture inducing acute thrombosis, arterial dissection or distal embolism.

Although the present invention has been described herein by way of preferred embodiments thereof, it can be modified without departing from the spirit, scope and nature of the subject invention, as defined in the appended claims.

List of References

1. A. J. Pfister, The safety of CABG without cardiopulmonary bypass, Ann Thorac Surg 64:590-1.

2. R. Ascione, C. T. Lloyd, M. J. Underwood, A. A. Lotto, A. A. Pitsis, G. D. Angelini, Economic outcome of off-pump coronary artery bypass surgery: a prospective randomized study, Ann Thorac Surg 68:2237-42.

3. V. G. Trapp, R. Bisarya, Placement of coronary artery bypass graft without pump oxygenator, Ann Thorac Surg 19 (1975) 1-9.

4. O. Chavanon, L. P. Perrault, P. Menasché, M. Carrier, P. M. Vanhoutte, Endothelial effects of hemostatic devices for continuous cardioplegia or minimally invasive operations. Updated in 1999, Ann Thorac Surg 68 (1999) 1118-1120.

5. R. G. Demaria, S. Fortier, O. Malo, M. Carrier, L. P. Perrault, Influence of intracoronary shunt size on coronary endothelial function during OPCAB, Heart Surg Forum 5 (2002) S165.

6. H. B. Hangler, K. Pfaller, H. Antretter, O. E. Dapunt, J. O. Bonatti, Coronary endothelial injury after local occlusion on the human beating heart, Ann Thorac Surg 71 (2001) 122-127.

7. R. G. Demaria, S. Fortier, M. Carrier, L. P. Perrault, Early multifocal stenosis after coronary artery snaring during OPCAB in a diabetic patient, J Thorac Cardiovasc Surg 122 (2001) 1044-1045.

8. O. E. Dapunt, M. R. Raji, S. Jeschkeit, S. Dhein, F. Kuhn-Régnier, M. Südkamp, J. H. Fisher, U. Mehlhorn, Intracoronary shunt insertion prevents myocardial stunning in a juvenile porcine MIDCAB model absent of coronary artery disease, Eur J Cardiothorac Surg 15 (1999) 173-179.

9. V. Lucchetti, F. Capasso, M. Caputo, G. Grimaldi, M. Capece, G. Brando, S. Caprio, G. D. Angelini, Intracoronary shunt prevents left ventricular function impairment during beating heart coronary revascularization, Eur J Cardiothorac Surg 15 (1999) 255-259.

10. M. Ricci, H. L. Karamanoukian, G. D'Ancona, J. DeLaRosa, R. L. Karamanoukian, S. Choi, J. Bergsland, T. A. Salemo, Survey of resident training in beating heart operations, Ann Thorac Surg 70 (2000) 479-482.

11. P. M. Vanhoutte, H. Shimokawa, Endothelium-derived relaxing factor and coronary vasospasm, Circulation 80 (1989) 1-9.

12. L. P. Perrault, N. Desjardins, C. Nickner, P. Geoffroy, J. F. Tanguay, M. Carrier, Effects of occlusion devices for minimally invasive coronary artery bypass surgery on coronary endothelial function of atherosclerotic arteries, Heart Surg Forum 3 (2000) 287-292.

13. H. Shimokawa, L. L. Aarhus, P. M. Vanhoutte, Porcine coronary arteries with regenerated endothelium have a reduced endothelium-dependent responsiveness to aggregating platelets and serotonin. Circ Res 61 (1987) 256-270.

1. An intracoronary shunt acting to retain a blood vessel open and allow blood flow therethrough during a coronary procedure comprising:
a body having a blood flow lumen therethrough, the blood flow lumen having first and second openings for passing blood from one side of an opening in the blood vessel to a second side of the opening in the blood vessel; and
an occluding member attached to the body and being sized and configured to occlude the blood vessel on a single side of the opening in the blood vessel.

2. An intracoronary shunt as defined in claim 1, in which said occluding member is slightly greater in diameter than the exterior diameter of said body.

3. An intracoronary shunt as defined in claim 1, wherein said body is transparent.

4. An intracoronary shunt as defined in claim 1, wherein said occluding member at the end of said body is integral with it.

5. An intracoronary shunt as defined in claim 1, wherein the diameter of said body is selected so as to be slightly inferior to the diameter of said blood vessel.

6. An intracoronary shunt as defined in claim 1 which is constructed from a biocompatible material selected from the group consisting of polyethylene, polyurethane, nylon, silicone, or other suitable single or composite material.

7. An intracoronary shunt as defined in claim 1, wherein said coronary procedure is an anastamosis.

8. An intracoronorary shunt acting to retain a blood vessel open and allow blood flow therethrough during a coronary procedure comprising a T-shaped shunt adapted to be inserted and removed through an incision in the blood vessel and including:
an outer elongated primary perfusion tube having a first open end, a second open end and a central passage extending between and interconnecting said open ends;
an inner secondary perfusion tube intersecting the primary perfusion tube and having a first open end and a second open end intersecting with the primary tube and having a central passage extending between said open ends; and
an enlarged occluding member adjacent one open end of the primary tube to seal the interior of the blood vessel, the primary perfusion tube passage allowing blood flow through the vessel during cardiac or vascular procedures.

9. An intracoronary shunt as defined in claim 8, wherein said secondary perfusion tube passage intersects the primary perfusion tube passage for fluid communication therewith.

10. An intracoronary shunt as defined in claim 9, in which said secondary perfusion tube allows connection to an appropriate blood supply.

11. An intracoronary shunt as defined in claim 8, in which said occluding member is slightly greater in diameter than the exterior diameter of said primary perfusion tube.

12. An intracoronary shunt as defined in claim 8, in which a Y-connector is inserted in the secondary perfusion tube to provide a direct line and a second line from the Y-connector for a needle-less valve.

13. An intracoronary shunt as defined in claim 8, wherein said primary and secondary perfusion tubes are unitary thin wall tubes, with the primary tube being of a slightly smaller exterior diameter than the interior diameter of the blood vessel.

14. An intracoronary shunt as defined in claim 8, wherein said primary and secondary perfusion tubes are transparent.

15. An intracoronary shunt as defined in claim 8, wherein said occluding member at the end of said primary perfusion tube is integral with said tube.

16. An intracoronary shunt as defined in claim 8, wherein said shunt provides a dual perfusion, completely open system.

17. An intracoronary shunt as defined in claim 8, wherein the diameter of said elongated primary perfusion tube is selected so as to be slightly inferior to the diameter of said blood vessel.

18. An intracoronary shunt as defined in claim 8 which is constructed from a biocompatible material selected from the group consisting of polyethylene, polyurethane, nylon, silicone, or other suitable single or composite material.

19. An intracoronary shunt as defined in claim 8, wherein said coronary procedure is an anastamosis.

20. A method for retaining blood flow through a blood vessel during coronary procedures on a patient, including the steps of locating the blockage in a blood vessel necessitating bypass surgery, making an incision adjacent the blockage, inserting an intracoronary shunt as defined in claim 1 into the blood vessel to retain the vessel open, said shunt having an occluding member at one end to prevent leaking of blood around the shunt, suturing a vein graft onto the outline of the incision, and gradually removing the intracoronary shunt as the suturing of the graft to the blood vessel is completed.

21. A method for retaining blood flow through a blood vessel during coronary procedures on a patient, including the steps of locating the blockage in a blood vessel necessitating bypass surgery, making an incision adjacent the blockage, inserting an intracoronary shunt as defined in claim 8 into the blood vessel to retain the vessel open, said shunt having an occluding member at one end to prevent leaking of blood around the shunt, suturing a vein graft onto the outline of the incision, and gradually removing the intracoronary shunt as the suturing of the graft to the blood vessel is completed.

22. A method as described in claim 21, wherein drugs may be inserted into the shunt via the secondary perfusion tube during the procedure.

23. A method as described in claim 21, wherein said secondary perfusion tube intersects and extends to the exterior of the patient's body from the primary perfusion tube for insertion of drugs or blood from a secondary source.

24. A method as described in claim 21, wherein additional blood may be pumped from another area of the patient's body or from an external pump through the secondary perfusion tube.

25. A method as described in claim 21, wherein the coronary bypass procedure is accomplished with thoracoscopic instruments involving several small ports resulting in a minimally invasive coronary bypass surgery.

26. A method as described in claim 25, wherein said intracoronary shunt is capable of insertion into the chest cavity of the patient through a small incision for endoscopic surgery.
